(19) United States
(12) Patent Application Publication
(10) Pub. No.: US 2016/0136351 A1
(43) Pub. Date: May 19, 2016

(54) IRRIGATOR FOR WASHING WHILE SURGERY OPERATION

(71) Applicant: Caleb Co., Ltd., Gangwon-do (KR)

(72) Inventor: Kyung-kil Yoo, Seoul (KR)

(73) Assignee: Caleb Co., Ltd., Gangwon-do (KR)

(21) Appl. No.: 14/937,880

(22) Filed: Nov. 11, 2015

(30) Foreign Application Priority Data
Nov. 13, 2014 (KR) 10-2014-0158357

(51) Int. Cl. A61M 3/02; A61B 17/88; A61M 39/22

(52) U.S. Cl. CPC A61M 3/0279; A61M 39/22; A61B 17/88; A61B 2217/007

(57) ABSTRACT

A surgical irrigator is disclosed. The surgical irrigator includes a body unit, a spray unit engaged with one end of the body unit, and a control module mounted to the body unit and configured to control an amount of a cleansing solution sprayed through the spray unit. The control module includes a body part having a through hole configured to allow the cleansing solution to pass therethrough, and a driving part disposed inside the body part and configured to control an opening degree of the through hole.

### CROSS-REFERENCE TO RELATED PATENT APPLICATION

This application claims the benefit of Korean Patent Application No. 10-2014-0158357, filed on Nov. 13, 2014, which is hereby incorporated by reference as if fully set forth herein.

### BACKGROUND OF THE INVENTION

1. Field of the Invention

The present invention relates to a surgical irrigator for spraying an aqueous liquid onto an affected part during a surgical operation.

2. Discussion of the Related Art

In total joint replacement, the surface of a joint bone needs partial removal and thus the removed bone and blood should be washed out with a cleansing solution. The cleansing solution is a saline solution and an irrigator is used to spray the cleansing solution during the washing.

Total joint replacement is a surgical procedure performed on patients with degenerative arthritis, avascular necrosis with the femoral head, rheumatoid arthritis, tuberculous or inflammatory arthritis, and traumatic arthritis in any joint such as the hip, knee, shoulder, or elbow, when the patients are not treated with medication, physical therapy, and other conservative management. Total joint replacement seeks to relieve pain and restore normal motion for the patients.

As is implied from the appellation, total joint replacement seems to be a surgery in which a bone of a patient is completely cut out and an artificial bone is implanted. However, this is a wrong operation. In fact, total joint replacement is a surgical procedure in which the surface of a diseased joint bone is partially removed and covered with a thin film of a special metal and then a special plastic or ceramic is inserted between the thin film-covered bone and a bone to thereby enable the joint to make a smoother motion, like a dental procedure in which a decayed part is removed from a tooth having a cavity and the cavity is capped with gold or platinum.

A conventional irrigator includes a body with a cleansing solution spray nozzle at its one end and a space in its inside. The body is formed of a flexible material so as to be readily deformed just by a force applied by a hand.

To inject a cleansing solution into the internal space of the body, air is discharged from the body by applying a force to the body. In this state, the cleansing solution spray nozzle is immersed in a cleansing solution reservoir and the cleansing solution is sucked into the body by releasing the force from the body.

With the cleansing solution injected into the internal space of the body, the cleansing solution spray nozzle is directed to an affected part on which a surgical operation will be performed. Subsequently, when a force is applied to the body, the cleansing solution from the body is sprayed onto the affected part and the affected part is cleansed with the sprayed cleansing solution.

When the affected part is cleansed, it is necessary to control the sprayed pressure of the cleansing solution. For example, foreign materials may need to be washed out by spraying the cleansing solution with a high or low pressure. However, it is not easy to control the sprayed pressure of the cleansing solution with the conventional irrigator (refer to Korean Registered Patent No. 10-0786325 entitled by Irrigator for Washing Skin While Surgery Operation).

### SUMMARY OF THE INVENTION

Accordingly, the present invention is directed to a surgical irrigator that substantially obviates one or more problems due to limitations and disadvantages of the related art.

An object of the present invention is to provide a surgical irrigator for facilitating control of a sprayed pressure of a cleansing solution that is sprayed onto an affected part.

Additional advantages, objects, and features of the invention will be set forth in part in the description which follows and in part will become apparent to those having ordinary skill in the art upon examination of the following or may be learned from practice of the invention. The objectives and other advantages of the invention may be realized and attained by the structure particularly pointed out in the written description and claims hereof as well as the appended drawings.

To achieve these objects and other advantages and in accordance with the purpose of the invention, as embodied and broadly described herein, a surgical irrigator includes a body unit, a spray unit engaged with one end of the body unit, and a control module mounted to the body unit and configured to control an amount of a cleansing solution sprayed through the spray unit. The control module includes a body part having a through hole configured to allow the cleansing solution to pass therethrough, and a driving part disposed inside the body part and configured to control an opening degree of the through hole.

The driving part may be configured to make a reciprocal motion inside the body part and block the through hole.

The body part may include a body housing, a first through hole formed at one portion of the body housing, and a second through hole formed at the other portion of the body housing. The cleansing solution may be introduced through the first through hole and discharged through the second through hole.

The driving part may include a driving body configured to slide along a space inside the body housing, and a driving body hole penetrating through the driving body and configured to communicate with the first and second through holes during sliding.

An elastic member may be disposed at one end of the driving body and configured to be elastically deformed in a direction of the sliding, and a button part may be disposed at the other end of the driving body and configured to apply a force for sliding the driving body.

The driving part may be configured to switch from a first mode to a second mode through manipulation of the button part, the first mode being a mode in which the driving body hole communicates with the first and second through holes and the second mode being a mode in which the driving body hole does not communicate with the first and second through holes.

A part of the driving body hole may communicate with the first and second through holes in the second mode.

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 is a perspective view of a surgical irrigator according to an embodiment of the present invention;

FIG. 2 is a sectional view of the surgical irrigator illustrated in FIG. 1;

FIG. 3 is an enlarged conceptual view of a control module illustrated in FIG. 2;

FIG. 4 is a conceptual view of the control module illustrated in FIG. 3, viewed from a different direction;

FIG. 5 is a sectional view of the control module illustrated in FIG. 3; and

FIG. 6 is a conceptual view of a button part engaged with a driving part.

### DETAILED DESCRIPTION OF THE INVENTION

A surgical irrigator to which the present invention relates to will be described in detail with reference to the accompanying drawings. Like or similar reference numerals denote the same or similar components even in different embodiments. Once the same or similar components are described, the description is applied to them across the disclosure. Singular terms are intended to include plural forms, unless the context makes it clear that plural forms are not intended.

Terms ‘module’ and ‘unit’ used to name components are allocated or used interchangeably only in consideration of the easiness of writing the disclosure, without any distinguishing meaning or role.

As used in the description of the present invention, terms such as ‘first’ or ‘second’ may be used to describe various components, but do not limit such components. Such terms may be used to distinguish one component from another. For example, without departing from the scope of the present invention, a first component may be called a second component, and likewise, a second component may be called a first component.

If a component is said to be ‘connected with’ or ‘connected to’ another component, the component may be directly connected with, or connected to, the other component, or another component may exist in between. On the other hand, if a component is said to be ‘directly connected with’ or ‘directly connected to’ another component, it should be understood that no components exist in between.

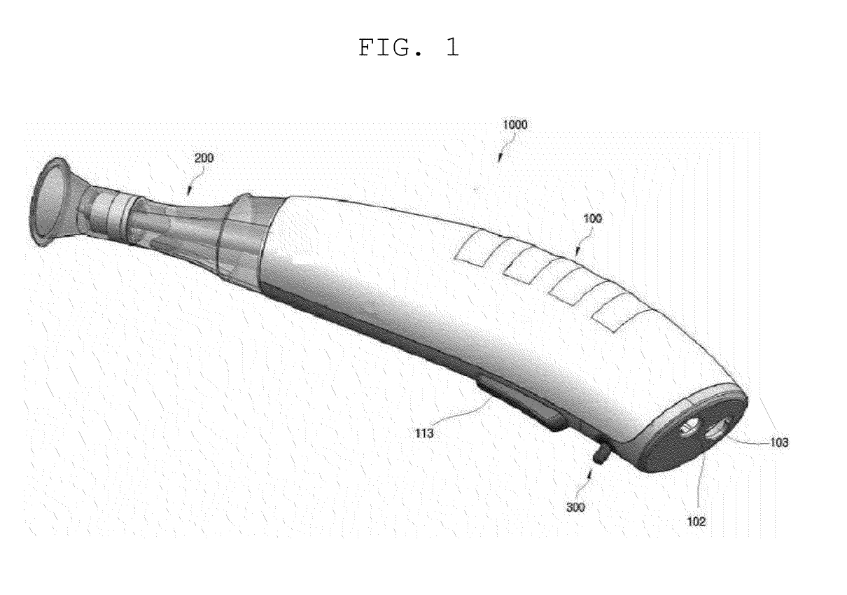

FIG. 1 is a perspective view of a surgical irrigator 1000 according to an embodiment of the present invention.

Referring to FIG. 1, the surgical irrigator 1000 includes a body unit 100 and a spray unit 200.

The spray unit 200 is detachably engaged with one portion of the body unit 100. The body unit 100 may include a switch 113 and a control module 300 configured to control a sprayed amount of water.

According to an embodiment of the present invention, the body unit 100 may include a first hole 102 and a second hole 103.

A pipe connected to a cleansing solution reservoir (not shown) outside the surgical irrigator 1000 may pass through the first hole 102. In general, a saline solution is used as a cleansing solution and thus the cleansing solution reservoir contains the saline solution. The cleansing solution reservoir may be a saline solution bottle called a fluid bottle.

A vacuum suction pipe connected to a vacuum pump (not shown) may pass through the second hole 103. The vacuum pump may be connected to the surgical irrigator 1000 of the present invention to suck out the cleansing solution sprayed onto a surgical site and blood and bone pieces from the surgical site by vacuum. The vacuum pump may be connected to the surgical irrigator 1000 through the second hole 103.

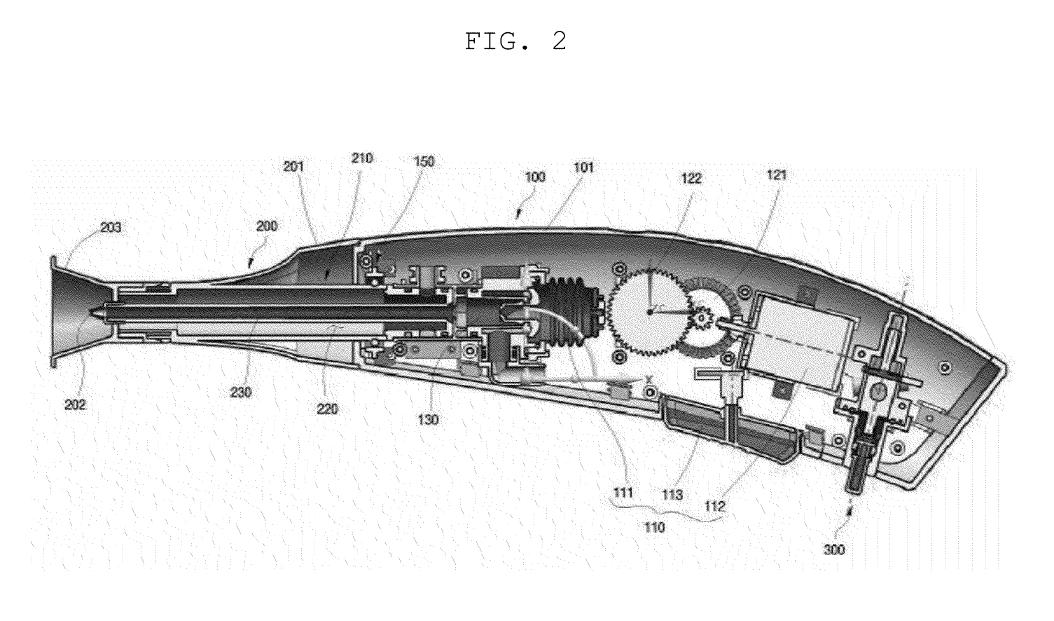

FIG. 2 is a sectional view of the surgical irrigator 1000 illustrated in FIG. 1.

Referring to FIG. 2, the surgical irrigator 1000 includes the body unit 100 and the spray unit 200.

The body unit 100 includes a housing 101 and various parts accommodated in the housing 101.

A cleansing solution supply module 110, the cleansing solution control module 300, and a driving force transfer module may be accommodated in the housing 101.

The cleansing solution supply module 110 may include an extension member 111, a driving motor 112, and the switch 113. The cleansing solution supply module 110 supplies the cleansing solution from the cleansing solution reservoir to the surgical irrigator 1000.

According to an embodiment of the present invention, the extension member 111 takes the form of an extendable bellow and is extended and shrunken by operations of the driving motor 112. The number of revolutions of the driving motor 112 is controllable by a user manipulation.

The driving force transfer module is configured to transfer a driving force of the driving motor 112 to the extension member 111. The driving force transfer module may include a first gear 121, a second gear 122, and a cam (not shown).

The first gear 121 may be a bevel gear that transfers rotation of an axis of the driving motor 112 to the second gear 122. The second gear 122 may be coupled to the cam (not shown). The cam is coupled to one end of the extension member 111 and transfers movement of the second gear 122 to the extension member 111. In other words, a rotation force of the driving motor 112 is transferred to the extension member 111 by means of the first gear 121, the second gear 122, and the cam and the extension member 111 is extended or shrunken by the transferred force.

The switch 113 controls a current applied to the driving motor 112. The switch 113 is a variable switch and may control the magnitude of the current applied to the driving motor 112. If the magnitude of the current applied to the driving motor 112 is changed, the number of revolutions of the driving motor 112 is also changed, thereby controlling an amount of the cleansing solution sprayed through the surgical irrigator 1000.

A cleansing solution chamber 130 serves as a temporary reservoir that temporarily contains the cleansing solution before it is sprayed onto a surgical site. The cleansing solution chamber 130 is connected to a cleansing solution spray tube 230. As the extension member 111 is extended and shrunken, a pressure is applied to the cleansing solution chamber 130 and the cleansing solution is sprayed through the spray tube 230 and then a spray nozzle 202.

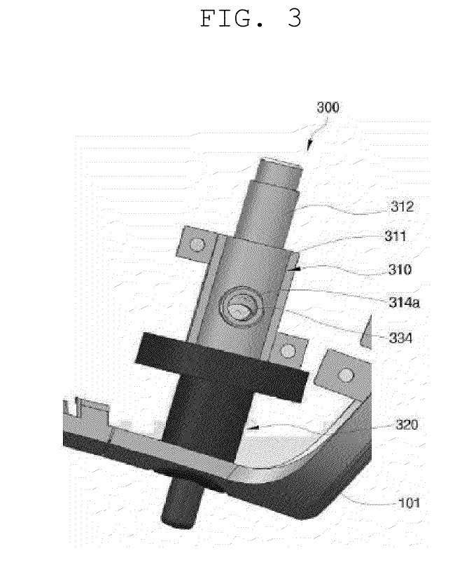

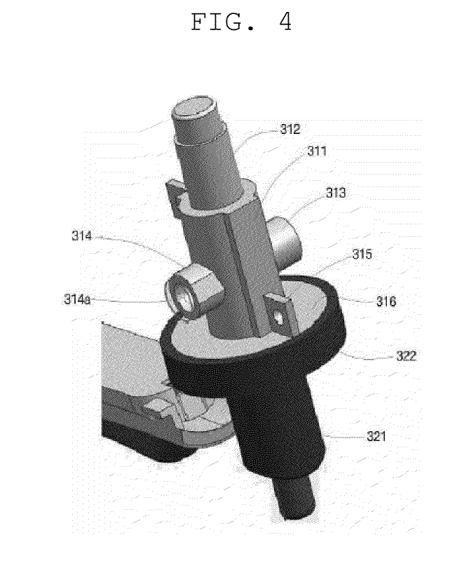

FIG. 3 is an enlarged conceptual view of the control module 300 illustrated in FIG. 2 and FIG. 4 is a conceptual view of the control module 300 illustrated in FIG. 3, viewed from a different direction.

Referring to FIGS. 3 and 4, the control module 300 includes a body part 310, a case part 320, a driving part 330, and a button part 340.

The body part 310 forms a housing for the control module 300. The body part 310 may include a first part 311 as a main housing and a second part 312 extended from the first part 311, for forming an additional space. According to an embodiment of the present invention, the first part 311 and the second part 312 may be incorporated into one large cylinder, rather than they are configured separately. While the first and second parts 311 and 312 are described as separate components for the convenience of description, the first and second parts 311 and 312 may not be configured separately or may be modified according to another embodiment of the present invention.

As illustrated in FIGS. 3 and 4, a first pipe 313 and a second pipe 314 may be formed in the first part 311.

The first pipe 313 is formed at one portion of the first part 311 and includes a first through hole (not shown). Like the first pipe 313, the second pipe 314 is formed at the other portion of the first part 311 and includes a second through hole 314a. The cleansing solution is introduced through the first through hole and discharged through the second through hole 314a. An amount of the cleansing solution discharged through the second through hole 314a is controlled according to the arrangement of the driving part 330 inside the body part 310.

A connection protrusion 315 may be formed in the first part 311, for connecting the control module 300 to the housing 101 of the body. A screw hole may be formed into the connection protrusion 315 and the body part 310 is fixed to the housing 101 by inserting a screw into the screw hole.

The body part 310 may be engaged with the case part 320. The case part 320 serves as a case surrounding the button part 340. The case part 320 protects the driving part 330 inside the control module 300, in engagement with the body part 310.

The case part 320 may include a first case part 321 and a second case part 322. A space in which the button part 340 is accommodated is formed inside the first case part 321 and a mounting space in which the body part 310 is mounted is formed in the second case part 322. The body part 310 may include a third part 316 which fits into the mounting space. The third part 316 is engaged with the second case part 322, forming the exterior of the control module 300.

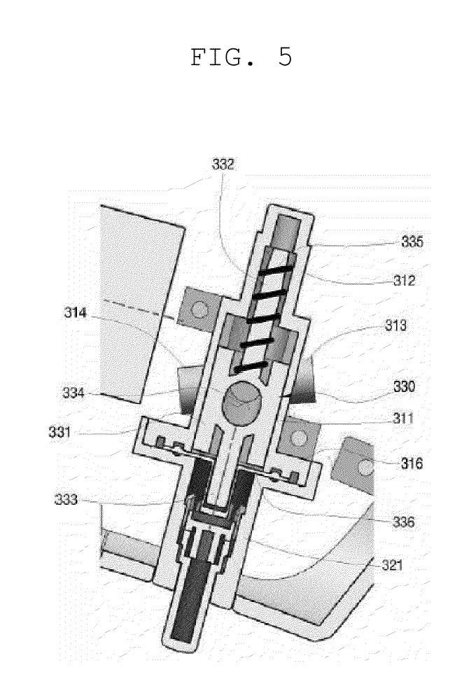

FIG. 5 is a sectional view of the control module 300 illustrated in FIG. 3.

Referring to FIG. 5, the driving part 330 and the button part 340 may be arranged inside the body part 310.

The driving part 330 may include a driving body 331, a first protrusion 333, a second protrusion 332, and a driving body hole 334. As illustrated in FIG. 5, the driving body 331 is configured to slide in contact with an inner surface of the first part 311 and the driving body hole 334 penetrates through the driving body 331. The first and second protrusions 333 and 332 may be formed on the bottom and top of the driving body 331, respectively.

Being engaged with the button part 340, the first protrusion 333 transfers a force applied to the button part 340 to the driving body 331. That is, a user may move the driving part 330 by manipulating the button part 340.

The second protrusion 332 is configured to fix a spring 335. A space for accommodating the second protrusion 332 is formed inside the second part 312 and the spring 335 is accommodated in the space. When the driving part 330 moves up and down, the spring 335 is elastically deformed, thus applying an elastic force to the driving part 330. The second protrusion 332 guides a direction in which the spring 335 is elastically deformed.

As illustrated in FIG. 5, a watertight member 336 may be interposed between the driving body 331 and the button part 340.

The watertight member 336 prevents leakage of the cleansing solution introduced in the control module 300 into the button part 340. With both ends of the watertight member 336 fixed between the third part 316 and the second case part 322, the center of the watertight member 336 is disposed between the first protrusion 333 and the button part 340. When the driving part 330 moves up and down, the center of the watertight member 336 may move up and down along with the driving part 330.

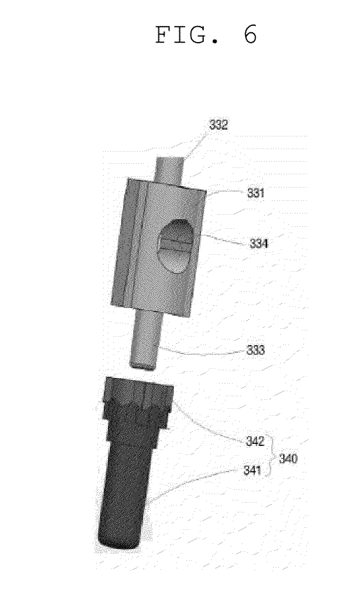

FIG. 6 is a conceptual view of the button part 340 engaged with the driving part.

Referring to FIG. 6, the button part 340 may include a first button body 341 exposed outward and a second button body 342 engaged with the first protrusion 333. A space for accommodating the second button body 342 may be formed inside the first button body 341. Further, the first and second button bodies 341 and 342 may be configured to make a relative motion with respect to each other. In other words, even though the second button body 342 moves up along with the driving body 331 by a force applied to the button part 340, the first button body 341 may be kept protruding outward from the housing 101.

As is apparent from the foregoing description, according to at least one of the above-described embodiments of the present invention, an amount of the cleansing solution introduced into a body unit may be primarily controlled by manipulating a button part. Therefore, a user can precisely control a sprayed amount and pressure of the cleansing solution according to a surgical situation, which makes it possible to build a safer and more efficient surgery environment.

The above-described surgical irrigator 1000 is not limited to the configurations and methods of the foregoing embodiments of the present invention. Rather, many variations and modifications may be made to the embodiments by selectively implementing the whole or part of each embodiment.

Those skilled in the art will appreciate that the present invention may be carried out in other specific ways than those set forth herein without departing from the spirit and essential characteristics of the present invention. The above embodiments are therefore to be construed in all aspects as illustrative and not restrictive. The scope of the invention should be determined by the appended claims and their legal equivalents, not by the above description, and all changes coming within the meaning and equivalency range of the appended claims are intended to be embraced therein.

What is claimed is:

1. A surgical irrigator comprising:
a body unit;
a spray unit engaged with one end of the body unit; and
a control module mounted to the body unit and configured to control an amount of a cleansing solution sprayed through the spray unit,
wherein the control module comprises:
a body part having a through hole configured to allow the cleansing solution to pass therethrough; and
a driving part disposed inside the body part and configured to control an opening degree of the through hole.

2. The surgical irrigator according to claim 1, wherein the driving part is configured to make a reciprocal motion inside the body part and block the through hole.

3. The surgical irrigator according to claim 2, wherein the body part comprises:
a body housing;
a first through hole formed at one portion of the body housing; and
a second through hole formed at the other portion of the body housing, and
wherein the cleansing solution is introduced through the first through hole and discharged through the second through hole.

4. The surgical irrigator according to claim 3, wherein the driving part comprises:
a driving body configured to slide along a space inside the body housing; and
a driving body hole penetrating through the driving body and configured to communicate with the first and second through holes during sliding.

5. The surgical irrigator according to claim 4, wherein an elastic member is disposed at one end of the driving body and configured to be elastically deformed in a direction of the sliding, and a button part is disposed at the other end of the driving body and configured to apply a force for sliding the driving body.

6. The surgical irrigator according to claim 5, wherein the driving part is configured to switch from a first mode to a second mode through manipulation of the button part, the first mode being a mode in which the driving body hole communicates with the first and second through holes and the second mode being a mode in which the driving body hole does not communicate with the first and second through holes.

7. The surgical irrigator according to claim 6, wherein a part of the driving body hole communicates with the first and second through holes in the second mode.
